(19) United States
(12) Patent Application Publication
(10) Pub. No.: US 2013/0123650 A1
(43) Pub. Date: May 16, 2013

(54) HEALTH MONITORING DEVICES

(71) Applicant: AcoMoTech, HsinChu City (TW)

(72) Inventors: Kurt CHIANG, HsinChu City (TW); Jung-Cheng LIN, HsinChu City (TW); Yi-Wei LIN, HsinChu City (TW); Shih-Heng CHENG, HsinChu City (TW); Yun-Yi TING, HsinChu City (TW)

(73) Assignee: ACOMOTECH, HsinChu City (TW)

(21) Appl. No.: 13/678,338

(22) Filed: Nov. 15, 2012

Related U.S. Application Data
(60) Provisional application No. 61/560,043, filed on Nov. 15, 2011.

(51) Int. Cl. A61B 5/0245

(52) U.S. Cl. CPC A61B 5/02455
USPC 600/502

(57) ABSTRACT

An electronic device which includes a detector, a counter and a processor. The detector is configured to detect a plurality of pulse signals and the counter is connected to the detector and configured to count the plurality of pulse signals. The processor is configured to receive the count from the counter and use the count to determine a heart rate, characterized in that the processor generates and send out an activation signal if the determined heart rate reaches or goes lower than a threshold of 60 beats per minute.

### CROSS REFERENCE TO RELATED APPLICATIONS

This application claims priority of U.S. Provisional Application No. 61/560,043 filed on Nov. 15, 2011 under 35 U.S.C. §119(e), the entire contents of all of which are hereby incorporated by reference.

### BACKGROUND OF THE INVENTION

The present invention generally relates to a health monitoring device and, more particularly, to a physiological signal monitoring device.

Products for health monitoring are prevalent nowadays. Various large-scale hospitals may be equipped with high-tech instruments to monitor a patient's condition. Domestic medical devices such as manometers, glucometers/glucose meters, theromometers and etc. may also be available in health care shops. However, most of such domestic medical devices cannot real-time monitor the condition of a user and timely send out an alert once any abnormal physiological signal is detected.

Therefore, it is desired in the market a health monitoring device that may keep detecting and analyzing physiological signals and alert people of the occurrence of abnormal event.

### BRIEF SUMMARY OF THE INVENTION

Examples of the present invention may provide an electronic device which includes a detector, a counter and a processor. The detector is configured to detect a plurality of pulse signals and the counter is connected to the detector and configured to count the plurality of pulse signals. The processor is configured to receive the count from the counter and use the count to determine a heart rate, characterized in that the processor generates and send out an activation signal if the determined heart rate reaches or goes lower than a threshold of 60 beats per minute.

Examples of the present invention may also provide an electronic device which includes a detector, a counter and a processor. The detector is configured to detect a plurality of pulse signals and the counter is connected to the detector and configured to count the plurality of pulse signals. The processor is configured to receive the count from the counter and use the count to determine a heart rate, characterized in that the processor generates and send out an activation signal if the determined heart rate reaches or goes over a threshold of 180 beats per minute for a certain interval.

Examples of the present invention may also provide an electronic device which includes a detector, a counter and a processor. The detector is configured to detect a plurality of pulse signals and the counter is connected to the detector and configured to count the plurality of pulse signals. The processor is configured to receive the count from the counter and use the count to determine a heart rate variability, which is represented by the changes of heart rate over an interval, characterized in that the processor generates and send out an activation signal if the determined heart rate variability reaches or goes lower than a threshold of −0.5.

Examples of the present invention may provide an electronic device which includes a detector, a counter and a processor. The detector is configured to detect a plurality of pulse signals and the counter is connected to the detector and configured to count the plurality of pulse signals. The processor is configured to receive the count from the counter and use the count to determine a heart rate, characterized in that the processor generates and send out an activation signal if the determined heart rate reaches or goes lower than a threshold of 60 beats per minute or the determined heart rate reaches or goes over a threshold of 180 beats per minute for a certain interval.

Additional features and advantages of the present invention will be set forth in portion in the description which follows, and in portion will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention as claimed.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, examples are shown in the drawings. It should be understood, however, that the invention is not limited to the precise arrangements and instrumentalities shown in the examples.

In the drawings:

FIG. 1A is a schematic top view of a first side of a monitoring device in accordance with an example of the present invention;

FIG. 1B is a schematic top view of a second side of a monitoring device in accordance with an example of the present invention;

FIG. 1C is a schematic perspective view of a monitoring device illustrated in FIGS. 1A and 1B; and

FIG. 2 is a schematic perspective view of a monitoring device in accordance with another example of the present invention.

### DETAILED DESCRIPTION OF THE INVENTION

Reference will now be made in detail to the present examples of the invention illustrated in the accompanying drawings. Wherever possible, the same reference numbers will be used throughout the drawings to refer to the same or like portions. It should be noted that the drawings are in greatly simplified form and are not to precise scale.

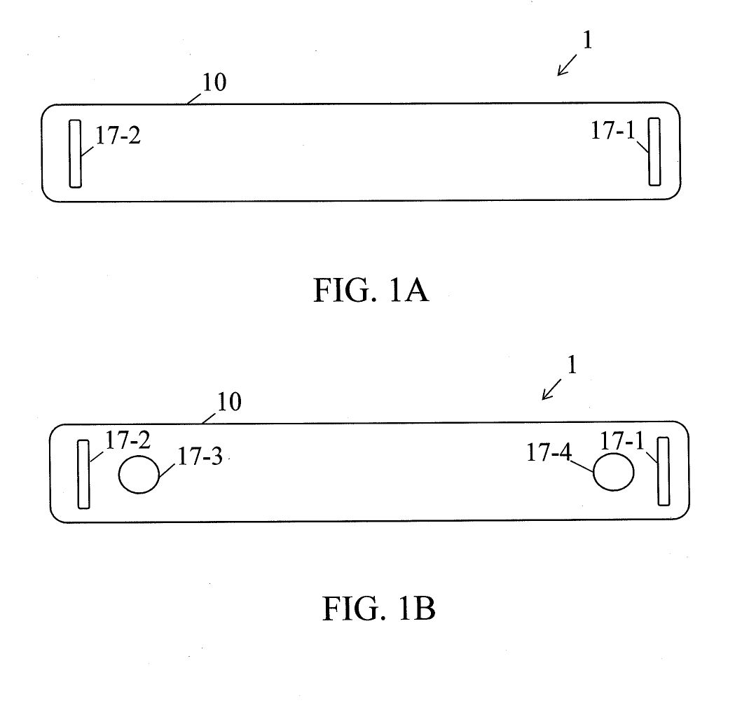

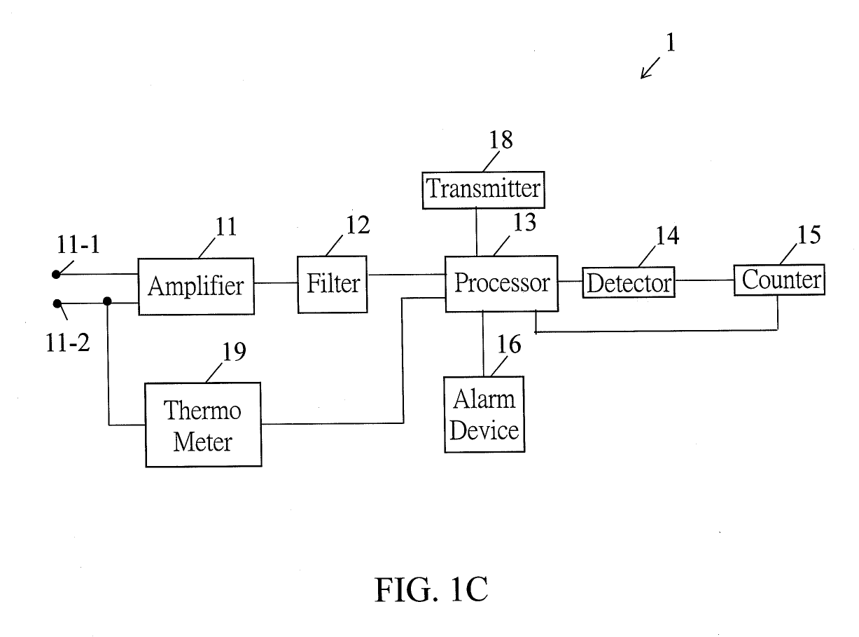

FIG. 1A is a schematic top view of a first side of a monitoring device in accordance with an example of the present invention. Referring to FIG. 1A, the monitor device 1 may include a body 10 which may have a first slot 17-1 and a second slot 17-2.

FIG. 1B is a schematic top view of a second side of a monitoring device in accordance with an example of the present invention, wherein the second side of the monitoring device is opposite to the first side. Referring to FIG. 1B, the body 10 may further include a first electrode 17-3 and a second electrode 17-4. Each of the first and second electrodes may be formed by but is not limited to one of the following electrically conductively materials: electrically conductive silicone, stainless steel. In another example, the body 10 may be formed by at least one of the aforesaid electrically conductively materials and the first electrode 17-3 and the second electrode 17-4 may be eliminated.

FIG. 1C is a schematic perspective view of a monitoring device illustrated in FIGS. 1A and 1B. Referring to FIG. 1C, the monitor device 1 may include a signal amplifier 11, a filter 12, a processor 13, a detector 14, a counter 15, an alarm device 16, a transmitter 18 and a thermo meter 19. The monitoring device 1 may also include a first terminal 11-1 and a second terminal 11-2. The first terminal 11-1 and the second terminal 11-2 may respectively, electrically connect to the first electrode 17-3 and the second electrode 17-4 as shown in FIG. 1B.

In one example, the amplifier 11 may be but is not limited to an electrocardiography (ECG) amplifier which may amplify signals received from the first terminal 11-1 and second terminal 11-2. The amplified signals may then be sent to the filter 12 to eliminate noises or interferences. The filtered signals may then be sent to the processor 13 and in turn the detector 14, which may be but is not limited to a QRS detector in the present example. The QRS detector 14 may identify a QRS complex and inform the counter 15 if any QRS complex is identified. The counter 15 may periodically send the count to the processor 13 which may determine whether or not to activate the alarm device 16 in accordance with the counting from the counter 15.

In one example, the processor 13 may use the count from the counter 15 to determine a heart rate (HR), means the number of heartbeats per unit of time, which may be expressed as beats per minute (bpm). If the determined HR reaches or goes lower than a threshold of 60 bpm, the processor 13 may activate the alarm device 16 at once and keep determining the HR. In another example, the processor 13 may also use the count from the counter 15 to determine heart rate variability (HRV), which is a physiological phenomenon where the time interval between heart beats varies and may be represented by a slope in the electrocardiogram (changes of HR over an interval). If the slope reaches or goes lower than a threshold of −0.5, the processor 13 may activate the alarm device 16 at once and keep determining the HR.

In another example, if the determined HR reaches or goes over a threshold of 180 bpm for a certain interval, for example, for consecutive three or five minutes, the processor 13 may activate the alarm device 16 at once and keep determining the HR.

Still referring to FIG. 1C, the thermo meter 19 may connect to the second terminal 11-2 and send the temperature-related signals to the processor 13. The processor 13 may determine whether or not to activate the alarm device 16 in accordance with the temperature-related signals from the thermo meter 19. In another example, the thermo meter 19 may connect to the first terminal 11-1. The processor 13 may also provide the transmitter 18 with signals associated with the count from the counter 15 and the temperature-related signals from the thermo meter 19.

In accordance with another example of the subject application, the amplifier 11, filter 12, detector 14, counter 15 and transmitter 18 may be implemented in hardware or software, in which the former may be more advantageous in view of operation speed while the latter may be more cost effective in view of design complexity. If implemented in hardware, the amplifier 11, filter 12, detector 14, counter 15 and transmitter 18 may be fabricated in an integrated chip (IC).

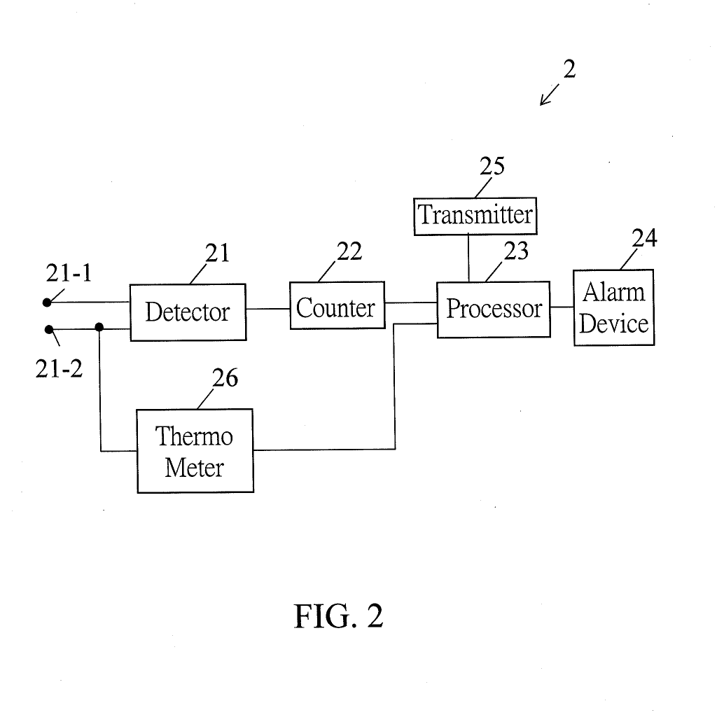

FIG. 2 is a schematic perspective view of a monitoring device in accordance with another example of the present invention. Referring to FIG. 2, the monitor device 2 may include a detector 21, a counter 22, a processor 23, an alarm device 24, a transmitter 25 and a thermo meter 26. The monitoring device 2 may also include a first terminal 21-1 and a second terminal 21-2. The monitor device 2 may be similar to the monitor device 1 as described and illustrated in FIG. 1C except that the amplifier 11 and filter 12 are eliminated and further replaced by the detector 24 and counter 25.

It will be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

Further, in describing representative examples of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. An electronic device comprising:
a detector configured to detect a plurality of pulse signals;
a counter connected to the detector and configured to count the plurality of pulse signals; and
a processor configured to receive the count from the counter and use the count to determine a heart rate, characterized in that the processor generates and send out an activation signal if the determined heart rate reaches or goes lower than a threshold of 60 beats per minute.

2. The electronic device of claim 1 further comprising an alarm device connected to the processor, wherein the alarm device receives the activation signal to generate an alarm signal.

3. The electronic device of claim 2 further comprising an amplifier and a filter, wherein the filter is connected between the processor and the amplifier.

4. The electronic device of claim 3 further comprising a thermo meter connected to the processor.

5. The electronic device of claim 1, wherein the processor generates and send out the activation signal if the determined heart rate reaches or goes over a threshold of 180 beats per minute for a certain interval.

6. The electronic device of claim 5, wherein the certain interval are three consecutive minutes.

7. The electronic device of claim 5, wherein the certain interval are five consecutive minutes.

8. The electronic device of claim 1, further comprising a transmitter connected to the processor.

9. An electronic device comprising:
a detector configured to detect a plurality of pulse signals;
a counter connected to the detector and configured to count the plurality of pulse signals; and
a processor configured to receive the count from the counter and use the count to determine a heart rate, characterized in that the processor generates and send out an activation signal if the determined heart rate reaches or goes over a threshold of 180 beats per minute for a certain interval.

10. The electronic device of claim 9, wherein the certain interval are three consecutive minutes.

11. The electronic device of claim 9, wherein the certain interval are five consecutive minutes.

12. The electronic device of claim 9, wherein the processor generates and sends out the activation signal if the determined heart rate reaches or goes lower than a threshold of 60 beats per minute.

13. An electronic device comprising:
a detector configured to detect a plurality of pulse signals;
a counter connected to the detector and configured to count the plurality of pulse signals; and
a processor configured to receive the count from the counter and use the count to determine a heart rate variability, which is represented by the changes of heart rate over an interval, characterized in that the processor generates and send out an activation signal if the determined heart rate variability reaches or goes lower than a threshold of −0.5.

14. An electronic device comprising:
a detector configured to detect a plurality of pulse signals;
a counter connected to the detector and configured to count the plurality of pulse signals; and
a processor configured to receive the count from the counter and use the count to determine a heart rate, characterized in that the processor generates and send out an activation signal if the determined heart rate reaches or goes lower than a threshold of 60 beats per minute or the determined heart rate reaches or goes over a threshold of 180 beats per minute for a certain interval.

15. The electronic device of claim 14, wherein the certain interval are three consecutive minutes.

16. The electronic device of claim 14, wherein the certain interval are five consecutive minutes.

17. The electronic device of claim 14 further comprising an amplifier and a filter, wherein the filter is connected between the processor and the amplifier.

18. The electronic device of claim 14 further comprising an alarm device connected to the processor, wherein the alarm device receives the activation signal to generate an alarm signal.

19. The electronic device of claim 14 further comprising a thermo meter connected to the processor.

20. The electronic device of claim 14 further comprising a transmitter connected to the processor.
